(19) United States
(12) Patent Application Publication
(10) Pub. No.: US 2026/0335559 A1
(43) Pub. Date: Oct. 29, 2026

(54) SINGLE-USE SYRINGE WITH AUTOMATIC LOCKING CLIP

(71) Applicant: Revital Healthcare (EPZ) Limited, Mombasa (KE)

(72) Inventors: Pramod Kumar NAYAK, Sikandra, Uttar Pradesh (IN); Harsh MEHTA, Leicester (GB)

(21) Appl. No.: 19/147,181

(22) PCT Filed: Jan. 12, 2023
(86) PCT No.: PCT/IB2023/050289

§ 371 Date: Jul. 10, 2025

(51) Int. Cl. A61M 5/315; A61M 5/50

(52) U.S. Cl. CPC A61M 5/31505; A61M 5/31511; A61M 5/5066; A61M 2005/5073

(57) ABSTRACT

A single-use syringe includes a barrel, a plunger, a gasket, and a clip. The plunger is inserted through a proximal end of the barrel. The plunger includes at least first, second, and third fins. The first and second fins have major planar surfaces that lie in a common plane. The clip slidably engages a radial edge of a third fin that has a major planar surface that is orthogonal to the common plane. The clip includes tabs that are configured to mechanically engage holes defined in the major planar surfaces of the first and second fins. The clip includes a pair of barbs that are configured to mechanically engage the barrel wall to resist a proximal movement of the clip with respect to the barrel and to slidably engage the barrel wall to allow a distal movement of the clip with respect to the barrel.

### RELATED APPLICATIONS

This application is a national phase entry of Application No. PCT/IB2023/050289 having an international filing date of Jan. 12, 2023 which is hereby incorporated by reference.

### TECHNICAL FIELD

This application relates generally to syringes.

### BACKGROUND

Medical syringes are used to deliver or collect fluids. Medical practitioners are instructed to use a syringe only once to prevent cross-contamination between patients. However, this single-use practice is not always followed.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

An aspect of the invention is directed to a single-use syringe comprising: a hollow cylindrical barrel having a proximal end and a distal end, the distal end having a narrowed tip opening; a plunger inserted through the proximal end of the hollow cylindrical barrel, the plunger comprising: a plurality of longitudinal fins that extend radially from an axis, the longitudinal fins extending to a proximal end of the plunger, each longitudinal fin including a respective major planar surface, the longitudinal fins including at least first, second, and third fins, wherein: the major planar surface of the first fin is aligned with the major planar surface of the second fin such that the major planar surfaces of the first and second fins lie in a common plane, the major planar surface of the third fin is orthogonal to the common plane, a first row of holes is defined in the major planar surface of the first fin, a second row of holes is defined in the major planar surface of the second fin, and the first and second rows of holes extend parallel to the axis. The syringe further comprises: a gasket attached to a distal end of the plunger; and a clip comprising: a body; a slot defined in the body, the slot configured to slidably engage a radial edge of the third fin; a pair of tabs configured to mechanically engage a pair of holes in the first and second rows of holes; and a pair of barbs at a proximal end of the clip, the barbs oriented away from the body and in a proximal direction, the barbs configured to mechanically engage an internal surface of the hollow cylindrical barrel to resist a movement of the clip in a proximal direction with respect to the hollow cylindrical barrel and to slidably engage the internal surface of the hollow cylindrical barrel to allow a movement of the clip in a distal direction with respect to the hollow cylindrical barrel.

In one or more embodiments, the axis is a first axis, and the holes in the first and second rows are evenly spaced and aligned with respect to a respective second axis that is orthogonal to the first axis. In one or more embodiments, a position of the holes in the first and second rows corresponds to a predefined volume of fluid in the syringe. In one or more embodiments, the gasket includes a gasket tip that is shaped to be inserted into the narrowed tip opening of the hollow cylindrical barrel.

In one or more embodiments, the syringe has a first configuration in which: the plunger is fully inserted into the hollow cylindrical barrel, the tabs are mechanically engaged with a pair of proximal end holes at a proximal end of the first and second rows of holes, and the clip is located between the distal and proximal ends of the hollow cylindrical barrel. In one or more embodiments, the syringe has a second configuration, the second configuration occurring after the first configuration, wherein in the second configuration: the plunger is partially retracted from the hollow cylindrical barrel, a position of the clip with respect to the hollow cylindrical barrel in the first and second configurations is the same, a position of the clip with respect to the plunger is closer to the distal end of the plunger in the second configuration than in the first configuration, the tabs are mechanically engaged with a respective pair of holes that correspond to the position of the clip with respect to the plunger, and a fluid reservoir is defined between the gasket and the distal end of the hollow cylindrical barrel, the fluid reservoir holding a fluid. In one or more embodiments, the syringe has a third configuration, the third configuration occurring after the second configuration, wherein in the third configuration: the plunger is fully retracted from the hollow cylindrical barrel, the position of the clip with respect to the hollow cylindrical barrel in the first, second, and third configurations is the same, the position of the clip with respect to the plunger is closer to the distal end of the plunger in the third configuration than in the second configuration, the clip positioned adjacent to a flange at the distal end of the plunger, the tabs are mechanically engaged with a pair of distal end holes at a distal end of the first and second rows of holes, and a volume of the fluid reservoir is larger in the third configuration than in the second configuration.

In one or more embodiments, the syringe has a fourth configuration, the fourth configuration occurring after the third configuration, wherein in the fourth configuration: the plunger is partially inserted into the hollow cylindrical barrel, the tabs are mechanically engaged with the pair of distal end holes, the position of the clip with respect to the hollow cylindrical barrel is closer to the distal end of the hollow cylindrical barrel in the fourth configuration than in the first, second, and third configurations, and the volume of the fluid reservoir is smaller in the fourth configuration than in the third configuration such that some of the fluid flows out of the fluid reservoir through the narrowed tip opening of the hollow cylindrical barrel. In one or more embodiments, has a fifth configuration, the fifth configuration occurring after the third or fourth configuration, wherein in the fifth configuration: the plunger is fully inserted into the hollow cylindrical barrel, the tabs are mechanically engaged with the pair of distal end holes, the clip is positioned adjacent to the flange at the distal end of the plunger, and the gasket is in physical contact with the distal end of the hollow cylindrical barrel, a position of the gasket causing the fluid to flow out of the fluid reservoir through the narrowed tip opening of the hollow cylindrical barrel. In one or more embodiments, the fifth configuration is a locked configuration in which: the flange prevents the clip from sliding in the distal direction with respect to the plunger, the pair of barbs resists the movement of the clip in the proximal direction with respect to the hollow cylindrical barrel, and the position of the gasket prevents a movement of the plunger in the distal direction with respect to the hollow cylindrical barrel, and a mechanical engagement of the tabs with the pair of distal end holes causes a resistance in the movement of the plunger in the proximal direction with respect to the hollow cylindrical barrel.

In one or more embodiments, the syringe has a sixth configuration, the sixth configuration occurring after the fifth configuration, wherein in the sixth configuration the plunger is broken at a predetermined mechanical failure point of the plunger, the break caused by a distal force on the plunger.

In one or more embodiments, an orientation notch is defined in the major planar surface of the third fin, the orientation notch configured to optically distinguish the third fin from the first and second fins.

In one or more embodiments, the common plane is a first plane, and the longitudinal fins include a fourth fin, the major planar surface of the fourth fin aligned with the major planar surface of the third fin such that the major planar surfaces of the third and fourth fins lie in a second plane that is orthogonal to the first plane. In one or more embodiments, an orientation notch is defined in the major planar surface of the third fin, an orientation gap is defined in the major planar surface of the fourth fin, and the orientation notch and the orientation gap configured to optically distinguish the third and fourth fins, respectively, from the first and second fins and to optically distinguish the third fin from the fourth fin.

In one or more embodiments, the tabs extend in the proximal direction and towards the first and second fins.

Another aspect of the invention is directed to a method of using a single-use syringe, comprising: placing the syringe in a fully retracted configuration, the syringe comprising: a hollow cylindrical barrel having a proximal end and a distal end, the distal end having a narrowed tip opening; a plunger inserted through the proximal end of the hollow cylindrical barrel, the plunger comprising: a plurality of longitudinal fins that extend radially from an axis, the longitudinal fins extending to a proximal end of the plunger, each longitudinal fin including a respective major planar surface, the longitudinal fins including at least first, second, and third fins, wherein: the major planar surface of the first fin is aligned with the major planar surface of the second fin such that the major planar surfaces of the first and second fins lie in a common plane, the major planar surface of the third fin is orthogonal to the common plane, a first row of holes is defined in the major planar surface of the first fin, a second row of holes is defined in the major planar surface of the second fin, and the first and second rows of holes extend parallel to the axis. The syringe further comprises a gasket attached to a distal end of the plunger; and a clip comprising: a body; a slot defined in the body, the slot configured to slidably engage a radial edge of the third fin; a pair of tabs configured to mechanically engage a pair of holes in the first and second rows of holes; and a pair of barbs at a proximal end of the clip, the barbs oriented away from the body and in a proximal direction, the barbs configured to mechanically engage an internal surface of the hollow cylindrical barrel to resist a movement of the clip in a proximal direction with respect to the hollow cylindrical barrel and to slidably engage the internal surface of the hollow cylindrical barrel to allow a movement of the clip in a distal direction with respect to the hollow cylindrical barrel. In the fully-retracted configuration: the plunger is fully retracted from the hollow cylindrical barrel, the clip is located between the distal and proximal ends of the hollow cylindrical barrel, the clip is positioned adjacent to a flange at the distal end of the plunger, the tabs are mechanically engaged within a pair of distal end holes at a distal end of the first and second rows of holes, and a fluid reservoir is defined between the gasket and the distal end of the hollow cylindrical barrel, the fluid reservoir holding a fluid. The method further comprises partially or fully inserting the plunger into the hollow cylindrical barrel; and while partially or fully inserting the plunger: flowing the fluid out of the fluid reservoir through the narrowed tip opening of the hollow cylindrical barrel; maintaining a mechanical engagement of the tabs with the pair of distal end holes; and sliding the barbs in the distal direction along the internal surface of the hollow cylindrical barrel, wherein the mechanical engagement of the tabs within the pair of distal end holes and an orientation of the barbs cause the syringe to transition automatically to a locked configuration after an insertion of the plunger into the hollow cylindrical barrel.

In one or more embodiments, the method further comprises, after the syringe is in the locked configuration: applying a separation force between the plunger and the hollow cylindrical barrel; mechanically engaging the barbs and the internal surface of the hollow cylindrical barrel, while applying the separation force, to resist a proximal movement of the clip with respect to the hollow cylindrical barrel; and mechanically engaging the tabs within the pair of distal end holes, while applying the separation force, to resist a proximal movement of the plunger with respect to the hollow cylindrical barrel.

In one or more embodiments, applying the separation force causes the plunger to break at a predetermined mechanical weakness point. In one or more embodiments, the method further comprises retracting the plunger from a fully-inserted position to the fully-retracted configuration; mechanically engaging the barbs and the internal surface of the hollow cylindrical barrel, while retracting the plunger, to resist a proximal movement of the clip with respect to the hollow cylindrical barrel; flexing the tabs, while retracting the plunger, to mechanically disengage the tabs from a pair of proximal end holes at a proximal end of the first and second rows of holes; sliding the radial edge of the third fin in the slot of the clip, while retracting the plunger, so that the tabs mechanically engage the pair of distal end holes; and flowing the fluid into the fluid reservoir, while retracting the plunger, from the narrowed tip opening.

In one or more embodiments, the method further comprises while retracting the plunger: mechanically engaging the tabs with a plurality of pairs of holes between the pair of proximal end holes and the pair of distal end holes; and flexing the tabs to mechanically disengage the tabs from each pair of holes between the pair of proximal end holes and the pair of distal end holes.

Another aspect of the invention is directed to a single-use syringe comprising: a hollow cylindrical barrel having a proximal end and a distal end, the distal end having a narrowed tip opening; a plunger inserted through the proximal end of the hollow cylindrical barrel, the plunger comprising: a plurality of longitudinal fins that extend radially from an axis, the longitudinal fins extending to a proximal end of the plunger, each longitudinal fin including a respective major planar surface, the longitudinal fins including at least first, second, and third fins, wherein: the major planar surface of the first fin is aligned with the major planar surface of the second fin such that the major planar surfaces of the first and second fins lie in a common plane, the major planar surface of the third fin is orthogonal to the common plane, a row of holes is defined in the major planar surface of the first fin, and the row of holes extends parallel to the axis. The syringe further comprises a gasket attached to a distal end of the plunger; and a clip comprising: a body; a slot defined in the body, the slot configured to slidably engage a radial edge of the third fin; a tab configured to mechanically engage a pair of holes in the first and second rows of holes; and a pair of barbs at a proximal end of the clip, the barbs oriented away from the body and in a proximal direction, the barbs configured to mechanically engage an internal surface of the hollow cylindrical barrel to resist a movement of the clip in a proximal direction with respect to the hollow cylindrical barrel and to slidably engage the internal surface of the hollow cylindrical barrel to allow a movement of the clip in a distal direction with respect to the hollow cylindrical barrel.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is a top view of a single-use syringe in a first configuration according to an embodiment.

FIG. 2 is a top view of the single-use syringe illustrated in FIG. 1 in a second configuration according to an embodiment.

FIG. 3 is a top view of the single-use syringe illustrated in FIG. 1 in a third configuration according to an embodiment.

FIG. 4 is a top view of the single-use syringe illustrated in FIG. 1 in a fourth configuration according to an embodiment.

FIG. 5 is a top view of the single-use syringe illustrated in FIG. 1 in a fifth configuration according to an embodiment.

FIG. 6 is a top view of the single-use syringe illustrated in FIG. 1 in an optional sixth configuration according to an embodiment.

FIG. 7 is a top view of a barrel according to an embodiment.

FIGS. 8 and 9 are top and side views, respectively, of a plunger according to an embodiment.

FIG. 10 is an end view looking from the distal end towards the proximal end of the plunger illustrated in FIGS. 8 and 9.

FIG. 11 is a top view of a plunger according to another embodiment.

FIG. 12 is a top view of a plunger according to another embodiment.

FIG. 13 is a bottom perspective view of a clip according to an embodiment.

FIG. 14 is a side view of the clip illustrated in FIG. 13.

FIG. 15 is a bottom perspective view of a clip according to another embodiment.

FIG. 16 is a flow chart of a method for using a single-use syringe according to an embodiment.

### DETAILED DESCRIPTION

A single-use syringe is disclosed that has an automatic locking clip that prevents syringe reuse. The syringe includes a barrel, a plunger, an automatic locking clip, and a gasket. The clip includes a pair of tabs that are configured to mechanically engage a set of holes defined in first and second fins of the plunger. The clip is slidably attached to a third fin on the plunger that is oriented orthogonally with respect to the first and second fins. The clip includes a pair of barbs that that are configured to mechanically engage the barrel wall to resist a proximal movement of the clip with respect to the barrel and to slidably engage the barrel to allow a distal movement of the clip with respect to the barrel. The clip is activated as soon as the plunger is depressed.

After the syringe is used and when the plunger is depressed, the barbs on the clip resist a proximal movement of the clip with respect to the barrel. The barbs also resist a proximal movement of the plunger with respect to the barrel since the plunger is mechanically coupled to the clip. The plunger can be configured to have a predetermined point or region where the plunger can break when a proximal and/or a distal force (e.g., an excessive force in the proximal and/or the distal directions) is applied to the plunger after the syringe is used. The plunger can break upon completion of the dose or after depressing the plunger at any depressed distance.

The single-use syringe can be an auto-disable syringe for fixed-dose immunization.

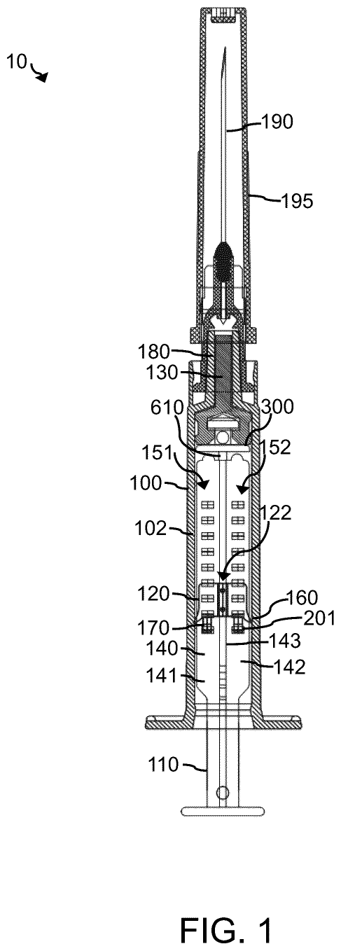

FIG. 1 is a top view of a single-use syringe 10 in a first configuration according to an embodiment. The syringe 10 includes a barrel 100, a plunger 110, a clip 120, and a gasket 130. The barrel 100 has a hollow cylindrical body 102 that is sized to slidably receive the plunger 110. The plunger 110 is fully inserted into the barrel 100 in the first configuration.

The plunger 110 includes a plurality of fins 140 that extend radially along the length of the insertable portion of the plunger 110. First and second rows of holes 151, 152 are formed in first and second fins 141, 142, respectively.

The clip 120 includes a pair of barbs 160 and a pair of tabs 170. The clip 120 also includes a slot 122 that slidably engages with and mounts the clip 120 on a third fin 143. The barbs 160 are oriented outwardly and in a proximal direction, away from the syringe tip 180, to mechanically engage the wall of the barrel 100. This orientation allows the clip 120 to slide in a distal direction, with respect to the barrel 100, towards the syringe tip 180, while preventing the sliding of the clip 120 in a proximal direction, with respect to the barrel 100, towards the opening of the barrel 100. The tabs 170 are axially aligned with the rows of holes 151, 152 and configured to be inserted into and/or mechanically engage within respective holes according to the relative position of the clip 120 and the plunger 110.

An optional needle 190 can be attached to the syringe tip 180. The needle 190 can be used to deliver a fluid (e.g., medication) to a patient or to extract a fluid sample from the patient. An optional needle cover 195 can cover the needle 190 until the syringe 10 is ready to be used.

The single-use syringe 10 is in a ready-to-use configuration, an as-assembled configuration, and/or an initial configuration in FIG. 1. In this configuration, the tabs 170 are inserted into the first hole 201 in each row of holes 151, 152. The first hole 201 is the hole at the proximal end of each row of holes 151, 152. The first holes 201 can alternately be referred to as proximal end holes.

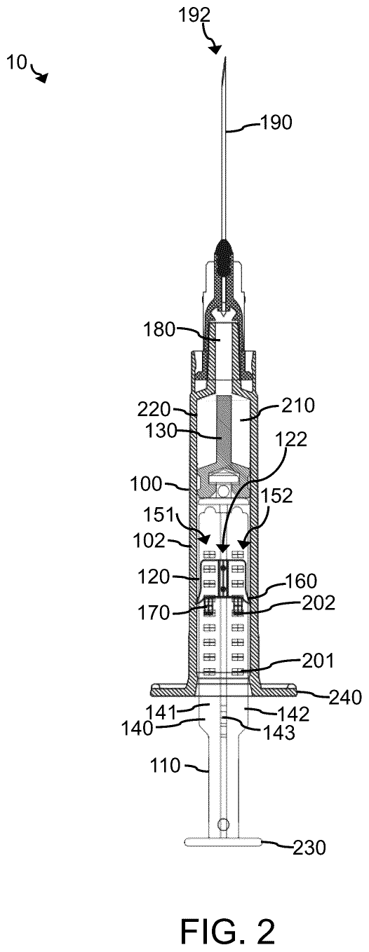

FIG. 2 is a top view of the single-use syringe 10 in a second configuration according to an embodiment. The second configuration can alternately be referred to as a partially-retracted configuration. In the second configuration, the plunger 110 has been retracted in a proximal direction relative to the barrel 100, compared to the first configuration of the syringe 10 in FIG. 1, such that the plunger 110 is in a partially retracted configuration. The plunger 110 can be retracted by applying a separation force between the plunger 110 and the barrel 100, such as by pulling on the plunger handle 230 while holding the barrel 100 and/or by pushing on the barrel 100, such as barrel flange 240, while holding the plunger 110.

Since the clip 120 is mounted on and mechanically coupled to the plunger 110 (e.g., via the tabs 170), the separation force that retracts the plunger 110 is also applied to the clip 120 in a proximal direction. The barbs 160 are configured to counter the proximal force on the plunger 110 to maintain the position of the clip 120 relative to the barrel 100 (e.g., compared to FIG. 1). Since the clip 120 remains stationary, the separation force causes the plunger 110 (e.g., the fin 143) to slide in a proximal direction with respect to the clip 120 as the plunger 110 is retracted in a proximal direction. From another perspective, the separation force causes the clip 120 to slide in a distal direction with respect to the plunger (e.g., the fin 143). When the clip 120 slides with respect to the plunger 110 (or vice versa), the tabs 170 mechanically engage the holes 151, 152 corresponding to the relative position of the clip 120 on the plunger 110. The tabs 170 are inserted into holes 202 in FIG. 2. In the transition between the first configuration (FIG. 1) and the second configuration (FIG. 2), as the clip slides 120 with respect to the plunger 110, the tabs 170 mechanically engage and/or are inserted into each pair of holes 151, 152 between holes 201 and holes 202, and then mechanically disengaged and/or released from each pair of holes 151, 152 between holes 201 and holes 202 as the tabs 170 flex. The pair of holes 202 is located distally compared to the pair of holes 201, such that the pair of holes 202 is located closer to the syringe tip 180 than the pair of holes 201.

In the partially-retracted configuration, a fluid reservoir 210 is formed at the distal end of the barrel 100. The fluid reservoir 210 is defined by the internal surface 220 of the barrel 100 (e.g., by an internal surface of a barrel wall), the gasket 130, and the syringe tip 180. Partially retracting the plunger 110 causes fluid to enter into the fluid reservoir 210 from the syringe tip 180 and through a channel 192 in the attached needle 190.

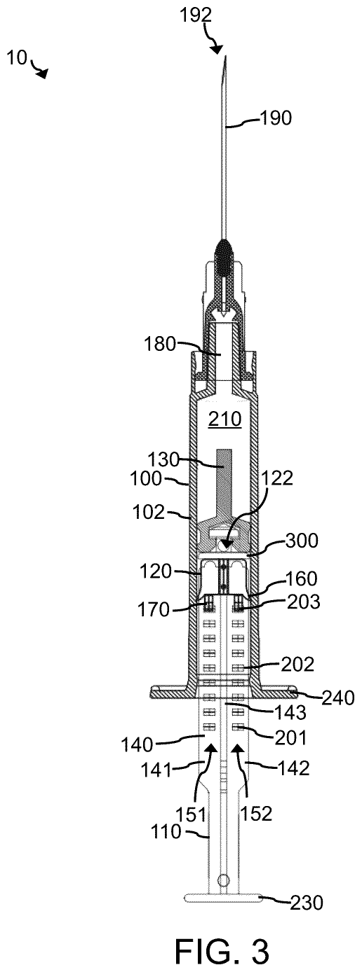

FIG. 3 is a top view of the single-use syringe 10 in a third configuration according to an embodiment. In the third configuration, the plunger 110 has been retracted in a proximal direction relative to the barrel 100, compared to the partially-retracted configuration illustrated in FIG. 2, such that the plunger 110 is in a fully retracted configuration.

As the plunger 110 is further retracted, the barbs 160 continue to counter the proximal force on the plunger 110 to maintain the position of the clip 120 relative to the barrel 100 (e.g., compared to FIGS. 1 and 2). Since the clip 120 remains stationary, the separation force causes the plunger 110 (e.g., the fin 143) to continue to slide in a proximal direction with respect to the clip 120 as the plunger 110 is further retracted in a proximal direction. From another perspective, the separation force causes the clip 120 to continue to slide in a distal direction with respect to the plunger (e.g., the fin 143). When the clip 120 slides with respect to the plunger 110 (or vice versa), the tabs 170 mechanically engage the holes 151, 152 corresponding to the relative position of the clip 120 on the plunger 110. When the plunger 110 is fully retracted, the tabs 170 are aligned with and inserted into and/or mechanically engaged within a pair of distal holes 203 in each row of holes 151, 152. The distal hole 203 is the last hole at the distal end of each row of holes 151, 152. In the transition between the second configuration (FIG. 2) and the third configuration (FIG. 3), as the clip slides 120 with respect to the plunger 110, the tabs 170 mechanically engage and/or are inserted into the holes 151, 152 between holes 202 and holes 203, and then mechanically disengaged and/or released from each pair of holes 151, 152 between holes 202 and holes 203 as the tabs 170 flex. Holes 203 are located distally compared to holes 201, 202, such that holes 202 are located between holes 201 and holes 203.

When the tabs 170 are inserted into holes 203, the distal end of the clip 120 is located adjacent to and/or in direct physical contact with a flange 300 at the distal end of the plunger 110. The flange 300 is configured to restrict a distal axial movement of the clip 120 relative to the plunger 110. When a user pulls on the plunger 110 in a proximal direction to further retract the plunger 110 from the barrel 100, the flange 300 prevents the clip 120 from sliding in a distal direction with respect to the plunger 110. From another perspective, the flange 300 prevents the plunger 110 from sliding in a proximal direction relative to the clip 120, such that any proximal force on the plunger 110 is also applied (e.g., via the flange 300) to the clip 120. Since the barbs 160 prevent or resist the plunger 110 from retracting in a proximal direction relative to the barrel 100 (e.g., as discussed above), the plunger 110 cannot be retracted further.

The volume of the fluid reservoir 210 is larger when the plunger 110 is in the fully retracted configuration (FIG. 3) than when it is in the partially-retracted configuration (FIG. 2). In some embodiments, the position of the holes 151, 152 correspond to pre-defined volumes of the fluid reservoir 210. For example, in an embodiment the volumetric capacity of the fluid reservoir 210 can be 0.5 ml in the fully retracted configuration. Each pair of holes 151, 152 can correspond to a pre-defined fraction (e.g., ⅛th) of the volumetric capacity of the fluid reservoir 210. For example, the first pair of holes 151, 152 at the proximal end (e.g., first holes 201) of the rows of holes can correspond to a fluid reservoir capacity of

$$\frac{0.5 ml}{8}$$

or 0.0625 ml, the second set of holes 151, 152, immediately adjacent to the first holes 201, can correspond to a fluid reservoir capacity of

$$\frac{2 \times 0.5 ml}{8}$$

or 0.120 ml, and so on. In general, the incremental pre-defined fraction of the volumetric capacity of each pair of holes 151, 152 can be equal to

$$\frac{1}{N - 1} \times {Vol}_{res}$$

where N is equal to the number of pairs of holes 151, 152 and Volᵣₑₛ is equal to the volumetric capacity of the fluid reservoir 210.

It is noted that in other embodiments the volumetric capacity of the fluid reservoir 210, in the fully retracted configuration of the plunger 110, can be greater than or less than 0.5 ml. For example, the volumetric capacity of the fluid reservoir 210, in the fully retracted configuration of the plunger 110, can be 0.1 ml, 0.25 ml, 0.3 ml, 0.5 ml, 1 ml, or 5 ml, or another volume in the fully retracted configuration.

Fully retracting the plunger 110 causes additional fluid to enter into the fluid reservoir 210 (e.g., through the syringe tip 180) compared to partially retracting the plunger 110 (FIG. 2).

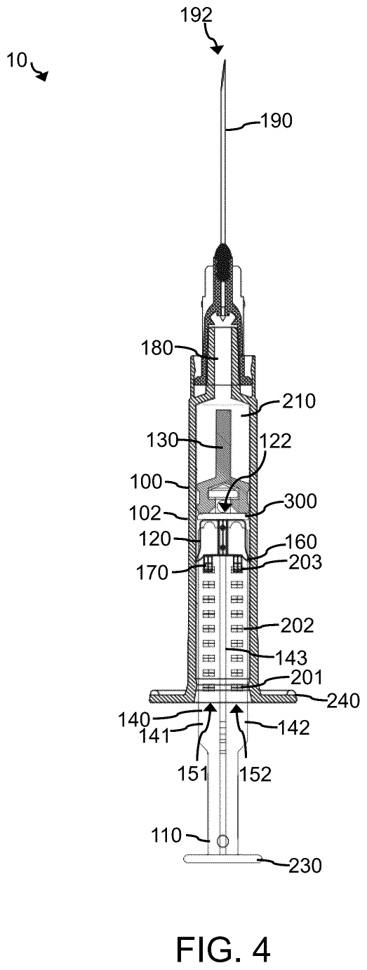

FIG. 4 is a top view of the single-use syringe 10 in a fourth configuration according to an embodiment. In this figure, the plunger 110 has been inserted in a distal direction relative to the barrel 100, compared to the fully-retracted configuration illustrated in FIG. 3, such that the plunger 110 is in a partially-inserted configuration. The plunger 110 can be inserted by applying an insertion force between the plunger 110 and the barrel 100, such as by pushing on the plunger handle 230 while holding the barrel 100 and/or by pulling on the barrel 100, such as the barrel flange 240, while holding the plunger 110.

Since the clip 120 is mounted on and mechanically coupled to the plunger 110 (e.g., via the tabs 170 in the distal pair of holes 203), the insertion force that inserts the plunger 110 is also applied to the clip 120 in a distal direction. The orientation of the barbs 160 allow the barbs 160 to slidably engage the wall of the barrel 100 to allow the clip 120 to move with the plunger 110 in the distal direction within the barrel 100. Thus, the plunger 110, the clip 120, and the gasket 130 are moved together in a distal direction within the barrel 100 compared to the fully-retracted configuration illustrated in FIG. 3.

As the plunger 110, clip 120, and gasket 130 move in a distal direction within the barrel 100, a portion of the fluid in the fluid reservoir 210 flows out of the syringe tip 180 and through the channel 192 in the needle 190.

After the plunger 110 is depressed, the barbs 160 resist a proximal movement of the clip 120 with respect to the barrel 100 to prevent reuse of the syringe 10.

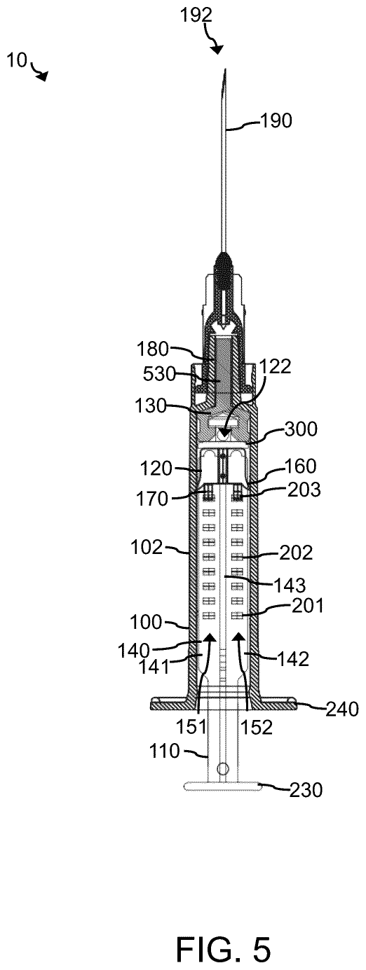

FIG. 5 is a top view of the single-use syringe 10 in a fifth configuration according to an embodiment. In this figure, the plunger 110 has been inserted in a distal direction relative to the barrel 100, compared to the partially-inserted configuration illustrated in FIG. 4, such that the plunger 110 is in a fully-inserted configuration.

To transition from the partially-inserted configuration (FIG. 4) to the fully-inserted configuration (FIG. 5), an insertion force is applied between the plunger 110 and the barrel 100. Since the clip 120 is mounted on and mechanically coupled to the plunger 110 (e.g., via the tabs 170 in the distal pair of holes 203), the insertion force that inserts the plunger 110 is also applied to the clip 120 in a distal direction. The orientation of the barbs 160 allow the barbs 160 to slidably engage the wall of the barrel 100 to allow the clip 120 to move with the plunger 110 in the distal direction within the barrel 100. Thus, the plunger 110, the clip 120, and the gasket 130 are moved together in a distal direction within the barrel 100 compared to the partially-retracted configuration illustrated in FIG. 4. The plunger 110, the clip 120, and the gasket 130 move in a distal direction within the barrel 100 until the gasket 130 contacts the distal end of the barrel 100.

As the plunger 110, clip 120, and gasket 130 move in a distal direction within the barrel 100, the remainder of the fluid in the fluid reservoir 210 (FIG. 4) flows out of the syringe tip 180 and through the channel 192 in the optional needle 190.

As can be seen, the gasket 130 is shaped to conform to the inside of distal end of the barrel 100. The gasket 130 also includes an optional gasket tip 530 that is shaped to be inserted into the inside (e.g., channel) of the syringe tip 180. The shape of the gasket 130 causes all or substantially of the fluid in the fluid reservoir 210 to flow out of the syringe tip 180. When the syringe 10 is used to deliver a therapeutic fluid, such as a medicine or vaccine, the shape of the gasket 130 can reduce waste that would occur with gaskets having other shapes that leave a small amount of fluid in the syringe (e.g., in the syringe tip 130).

After the plunger 110 is inserted partially or fully into the barrel 100 (e.g., as illustrated in FIGS. 4 and 5, respectively), the configuration of the clip 120 automatically causes the syringe 10 to be placed in a locked configuration in which the plunger 110 cannot be retracted from its current position within the barrel 100. Ideally, the syringe 10 would be discarded in this configuration since it cannot (or at least should not) be re-used. In order to re-use the syringe 10, the plunger 110 would need to be retracted in the barrel 100 for example to measure another dose of medication or to extract a fluid sample from another patient. However, the barbs 160 on the clip 120 are configured and/or oriented to prevent and/or resist the clip 120 from moving or sliding in a proximal direction within the barrel 100, as discussed. Since the clip 120 is mechanically coupled to the plunger 110 (e.g., via the tabs 170 and distal pair of holes 203), the barbs 160 on the clip 120 prevent and/or resist the plunger 110 from retracting.

It is noted that in the locked configuration the plunger 110 may be retracted within the barrel 100 if sufficient force is applied. For example, the plunger 110 may be retracted within the barrel 100 if the proximal force applied to the plunger is greater than the locking force of the barbs 160 on the barrel 100. In addition, the plunger 110 may be retracted within the barrel 100 if the tabs 170 mechanically fail in which case the clip 120 becomes mechanically disconnected from the plunger 110 which allows the plunger 110 to freely move within the barrel 100 relative to the clip 120.

As a further security feature, the plunger 110 can be formed with a predetermined mechanical failure point, region, or area. The predetermined mechanical failure point/region/area can be a point, region, or area on the plunger 110 that has a mechanical weakness compared to the rest of the plunger 110. The mechanical weakness can be formed by using less material in the predetermined mechanical failure point, region, or area compared to the rest of the plunger 110. Additionally or alternatively, the mechanical weakness can be formed using mechanical scores or perforations. Additionally or alternatively, the mechanical weakness can be formed by using a smaller amount of adhesive in the predetermined mechanical failure point, region, or area compared to the other portions of the plunger 110. The predetermined mechanical failure point, region, or area is configured to disable the syringe when excessive force is applied to the plunger 110, for example when attempting to retract the plunger 110 after use or after any distal movement of the plunger 110 in the barrel 100 (e.g., with respect to the initial or first configuration or another configuration). For example, the predetermined mechanical failure point, region, or area can be configured to disable the syringe 10 (e.g., to cause some of all of the plunger 110 to break) when a force is applied between the plunger 110 and the barrel 110, where the force can be about 16 N (newtons) to about 20 N, including about 17 N, about 18 N, about 19 N, and any value or range between any two of the foregoing forces. As used herein, “about” means plus or minus 10% of the relevant value.

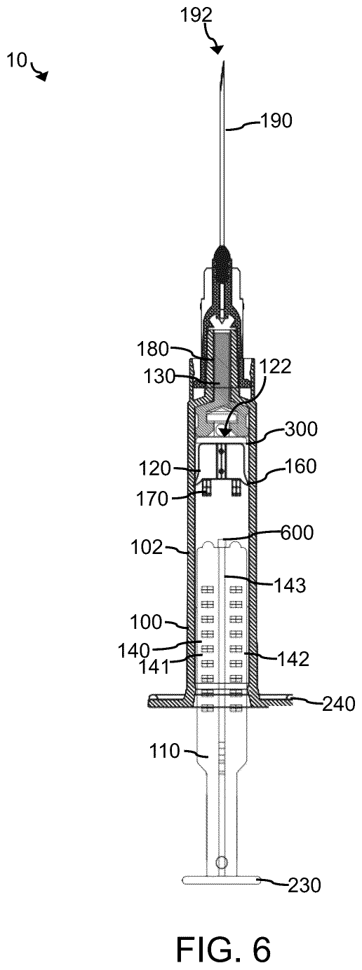

FIG. 6 is a top view of the single-use syringe 10 in an optional sixth configuration according to an embodiment. In this figure, the plunger 110 has been retracted within the barrel 100 after the plunger 110 was in a fully inserted configuration (FIG. 5). The proximal force on the plunger 110 causes the distal end 600 of the fins 140 to break away from the flange 300, causing the syringe 10 to be unusable. In this embodiment, the mechanical failure point/region/area is in the joint 610 (FIG. 1) between the fins 140 and the flange 300.

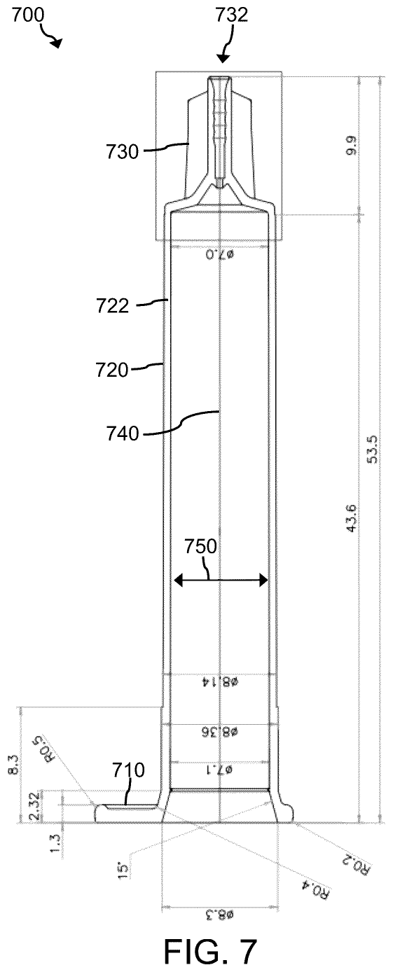

FIG. 7 is a top view of a barrel 700 according to an embodiment. The barrel 700 can be the same as or different than barrel 100.

The barrel 700 includes a flange 710, a hollow cylindrical body 720, and a narrowed tip 730. The flange 710 is located at the proximal end of the barrel 700. The tip 730 is located at the distal end of the barrel 700. The hollow cylindrical body 720 and the tip 730 are aligned along axis 740. The tip 730 includes a channel 732 through which fluid can flow into and out of the hollow cylindrical body 720. The tip 730 can be mechanically coupled to a needle. Additionally or alternatively, the tip 730 can include external threads to form a needle lock. The hollow cylindrical body 720 has an internal volume defined at least in part by a wall 722. The hollow cylindrical body 720 has an internal diameter 750 that is configured to allow the plunger to be received and to mechanically engage the barbs on the clip.

Various example dimensions and angles are illustrated in FIG. 7 for reference purposes only.

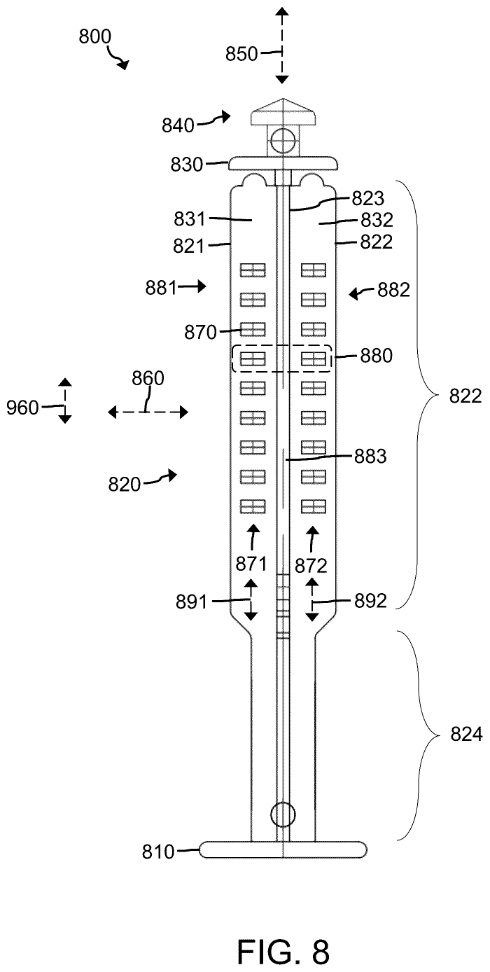

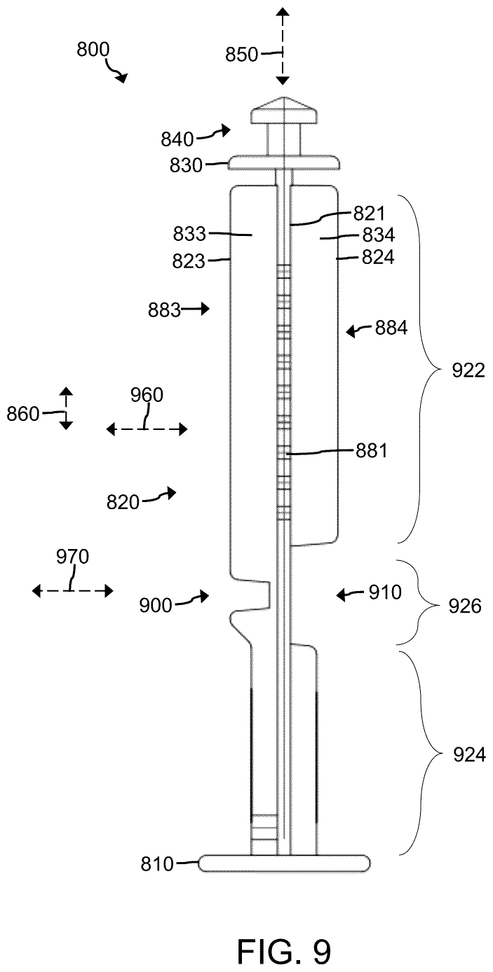

FIGS. 8 and 9 are top and side views, respectively, of a plunger 800 according to an embodiment. The plunger 800 can be the same as or different than plunger 110.

The plunger 800 includes a handle 810, a plurality of fins 820, a flange 830, and a tip 840. The handle 810 is located at the proximal end of the plunger 800. The tip 840 is located at the distal end of the plunger 800. The handle 810, fins 820, flange 830, and tip 840 are aligned and/or centered with respect to axis 850, which can be an axis of symmetry. The fins 820 extend radially from axis 850 and extend from the flange 830 to the handle 810 along the length of the plunger 800.

Opposing fins 821, 822 include a first section 822 and a second section 824. The width of the fins 821, 822, as measured with respect to axis 860, is larger in the first section 822 than in the second section 824. The combined width of the first section 822 of fins 821, 822 is smaller or slightly smaller than the internal diameter 750 of the hollow cylindrical body 720 (FIG. 7) of the barrel 700 to allow the first section 822 of the fins 821, 822 to fit therein. The length of the first section 822, as measured with respect to axis 850, is approximately equal to the length of the hollow cylindrical body 720 of the barrel 700, as measured with respect to axis 740. The first section 822 can be an insertable section of the fins 821, 822.

The narrowed width of the second section 824 of the fins 821, 822 resembles a shaft that the user can hold along with the handle 810. In another embodiment, the fins 821, 822 do not include the second section 824 in which case the fins 821, 822 can have a uniform width along some or all of their length, the uniform width being smaller or slightly smaller than the internal diameter 750 of the hollow cylindrical body 720.

Opposing fins 823, 824 include a first section 922 (FIG. 9) and a second section 924. The width of the fins 823, 824, as measured with respect to axis 960, is larger in the first section 922 than in the second section 924. Axes 850, 860, and 960 are orthogonal with respect to one another. The combined width of the first section 922 of fins 823, 824 is smaller or slightly smaller than the internal diameter 750 of the hollow cylindrical body 720 (FIG. 7) of the barrel 700 to allow the first section 922 of the fins 823, 824 to fit therein. The combined width of the first section 922 of fins 823, 824 can be about equal to the combined width of the first section 922 of fins 821, 822. The length of the first section 922, as measured with respect to axis 850, can be about equal to or smaller than the length of the first section 822. Fin 824 can be optional in some embodiments.

Each fin 821-824 has a respective radial edge 881-884 that has a surface that is orthogonal to the major planar surface of the respective fin 821-824. For example, fin 821 has a radial edge 881 that has a surface that is orthogonal to the major planar surface of fin 821. Fin 822 has a radial edge 882 that has a surface that is orthogonal to the major planar surface of fin 822. Fin 823 has a radial edge 883 that has a surface that is orthogonal to the major planar surface of fin 823. Fin 824 has a radial edge 884 that has a surface that is orthogonal to the major planar surface of fin 824. Each radial edge 881-884 extends along the length of the respective fin 821-824, the length measured with respect to axis 850. The width of each radial edge defines the thickness of the respective fin 820. For example, the width of radial edge 883, as measured with respect to axis 860, defines the thickness of fin 823.

The radial edge 883 of fin 823 can be configured and sized to receive and/or mechanically engage with a slot (e.g., slot 122) on the clip to allow the clip to be mounted (e.g., slidably mounted) thereon. Alternatively, the radial edge 884 of fin 824 can be configured and sized to receive and/or mechanically engage with a slot (e.g., slot 122) on the clip to allow the clip to be mounted (e.g., slidably mounted) thereon.

The narrowed width of the second section 924 of the fins 823, 824 resembles a shaft, for example in combination with the second section 824 of fins 821, 822, that the user can hold along with the handle 810. In another embodiment, the fins 823, 824 do not include the second section 924 in which case the fins 823, 824 can have a uniform width along some or all of their length, the uniform width being smaller or slightly smaller than the internal diameter 750 of the hollow cylindrical body 720.

An orientation section 926 of fins 823, 824 is defined between the first and second sections 922, 924. One or more orientation features defined in the orientation section 926 can be automatically detected by a machine (e.g., through sensors such as optical sensors) to install the clip 120 on a predetermined fin 820. The clip 120 is preferably installed and/or mounted on the radial edge 883 of fin 823.

In the orientation section 926, an orientation notch 900 is defined in fin 823 and an orientation gap 910 is defined in fin 824. The orientation notch 900 is located in a portion of fin 823 that has about the same width as the first section 922 of fin 823. The orientation notch 900 can alternately be described as being defined at the proximal end of the first section 922 of fin 823. The orientation gap 910 is defined in a portion of fin 824 that has about the same width as the first section 922 of fin 824. The orientation gap 910 can alternately be described as being defined at the proximal end of the first section 922 of fin 824 and/or between the first and second sections 922, 924 of fin 824. The orientation gap 910 is preferably aligned with and/or centered with respect to the notch 900, for example with respect to axis 970. Axis 970 is parallel to axis 960. The orientation gap 910 is wider, as measured with respect to axis 850, than the orientation notch 900.

A machine can use the orientation features (e.g., orientation notch 900 and orientation gap 910) to automatically distinguish, such as through optical sensors, between fins 821, 822 and fins 823, 824. The machine can also use the features (e.g., notch 900 and gap 910) to automatically distinguish, such as through optical sensors, between fin 823 and fin 824. For example, the difference in the width of the notch 900 and gap 910 can be used to optically distinguish fin 823 from fine 824. An assembly machine can be mechanically configured to align the plunger in a certain direction to ensure the clip is assembled on the correct fins.

Each fin 821-824 has a respective major planar surface 831-834. The major planar surfaces 831, 832 are parallel to each other and lie in a first common plane defined by axes 850, 860. The major planar surfaces 833, 834 of the fins 823, 824 are parallel to one another and orthogonal to the major planar surfaces 831, 832. The major planar surfaces 833, 834 lie in a second common plane defined by axes 850, 960.

A respective row of holes 871, 872 is defined in each planar surface 831, 832. The holes 870 in each row 871, 872 are aligned along a respective axis 891, 892, which is parallel to axis 850. The holes 870 are preferably evenly spaced along each axis 891, 892 in each row 871, 872. The aligned holes 870 in the first and second rows 871, 872 form pairs or sets 880 of holes, which can alternately be referred to as columns of holes. The spacing and/or position of the holes 870 can correspond to volumetric equivalents and/or volumetric graduations of the fluid reservoir 210 (e.g., FIG. 2), as described above. Each row 871, 872 preferably has the same number of holes 870. The holes 870 in each row 871, 872 are preferably aligned and/or centered with respect to a respective axis that is parallel to axis 860. The alignment of the holes 870 in each row 871, 872 can allow the tabs 170 (e.g., FIG. 1) on the clip 120 to mechanically engage within and/or to insert into the pairs/sets 880 of holes 870 in each row 871, 872 simultaneously.

The holes 870 are rectangular (e.g., in a cross section parallel to the planar surface 831, 832) in the embodiment illustrated in FIG. 8. In other embodiments, the holes 871 can be square, trapezoidal, triangular, oval, circular, or another shape.

In some embodiments, fins 821-824 can be integrally formed as a single structure, such as through molding or injection molding.

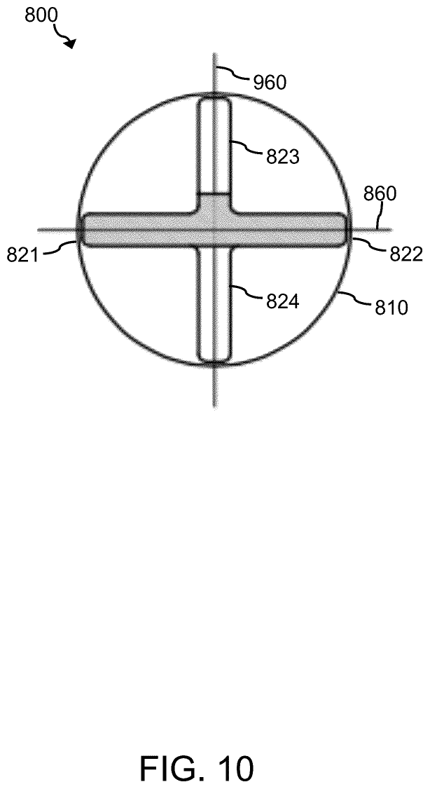

FIG. 10 is an end view looking from the distal end towards the proximal end of the plunger 800. The flange 830 and tip 840 are removed to further illustrate the relative orientation of the fins 821-824.

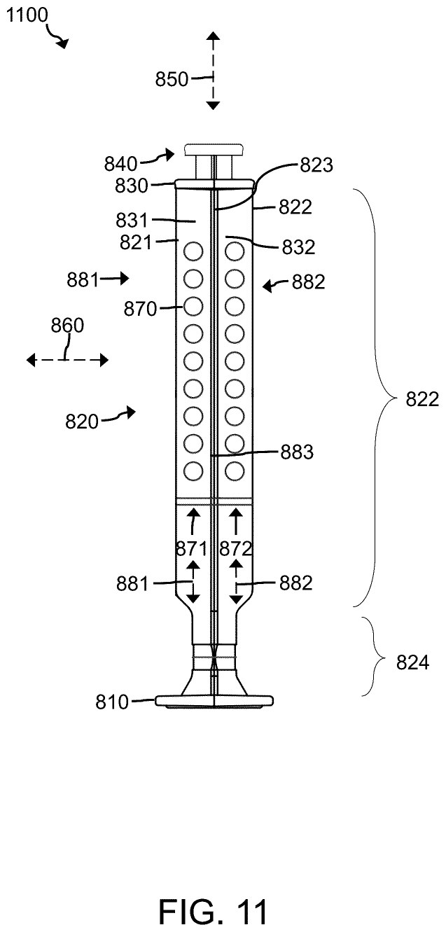

FIG. 11 is a top view of a plunger 1100 according to another embodiment. Plunger 1100 is the same as plunger 800 except that plunger 1100 includes circular holes (e.g., in a cross section parallel to the planar surface 831, 832). In addition, the length of the second section 824, as measured with respect to axis 850, is shorter in plunger 1100 than in plunger 800.

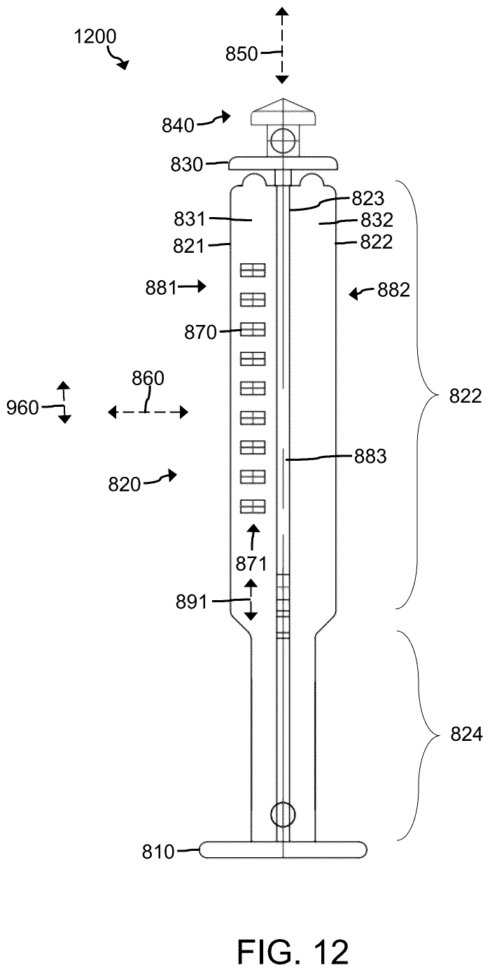

FIG. 12 is a top view of a plunger 1200 according to another embodiment. Plunger 1200 is the same as plunger 800 except that plunger 1200 includes only one row 871 of holes 870. The holes 870 are illustrated as rectangular (e.g., in a cross section parallel to the planar surface 831, 832) but can be another shape, as discussed above. The holes 870 are preferably evenly spaced and aligned with respect to axis 891.

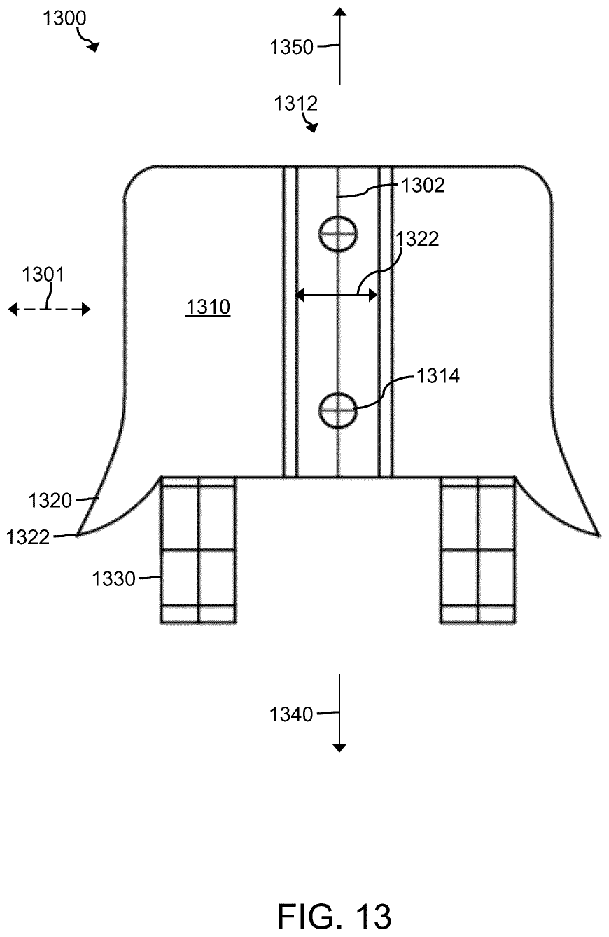

FIG. 13 is a bottom perspective view of a clip 1300 according to an embodiment. Clip 1300 can be the same as clip 120.

Clip 1300 includes a body 1310, a pair of barbs 1320, and a pair of tabs 1330. The body 1310 has a general rectangular shape but can be another shape such as an oval or square. A slot 1312 is defined in the back side of the body 1310 (facing away from the page in the bottom view illustrated in FIG. 13). The slot 1312 is configured to mount the body 1310 of the clip 1300 onto a radial edge of a fin (e.g., radial edge 883 of fin 823). For example, the slot 1312 can have a width 1322, as measured with respect to axis 1301, that is about the same as or slightly larger than the width of the radial edge of the fin to allow the slot 1312 to slidingly engage the radial edge of the fin. The slot 1312 extends the length of the body 1310 along axis 1302, which is orthogonal to axis 1301.

Dimples 1314 can be formed in the clip 1300. The dimples 1314 can reduce scratching of the barrel walls by the flat surfaces of the clip 1300 (e.g., on both slides of clip 1300). For example, without the dimples 1314, during uptake of the dose with the plunger, scratches would be formed and also when depressing the plunger more scratches would be formed. The scratches are undesirable as they can impact the integrity of the syringe. Therefore, the dimples 1314 (e.g., two dimples) can reduce the resistance on the barrel walls and prevent the barrel from getting damaged before use (e.g., administration of medicine).

Each barb 1320 has a point 1322 that is oriented outwardly (e.g., away from axis 1302) and in a proximal direction. The points 1322 of the barbs 1322 are oriented away from each other. The barbs 1300 are configured to mechanically engage the wall of the barrel when the clip 1312 is pushed in a proximal direction 1340 with respect to the barrel. The barbs 1300 are configured to flex inwardly and to slidably engage the internal surface of the barrel when the clip 1312 is pushed in a distal direction 1350 with respect to the barrel.

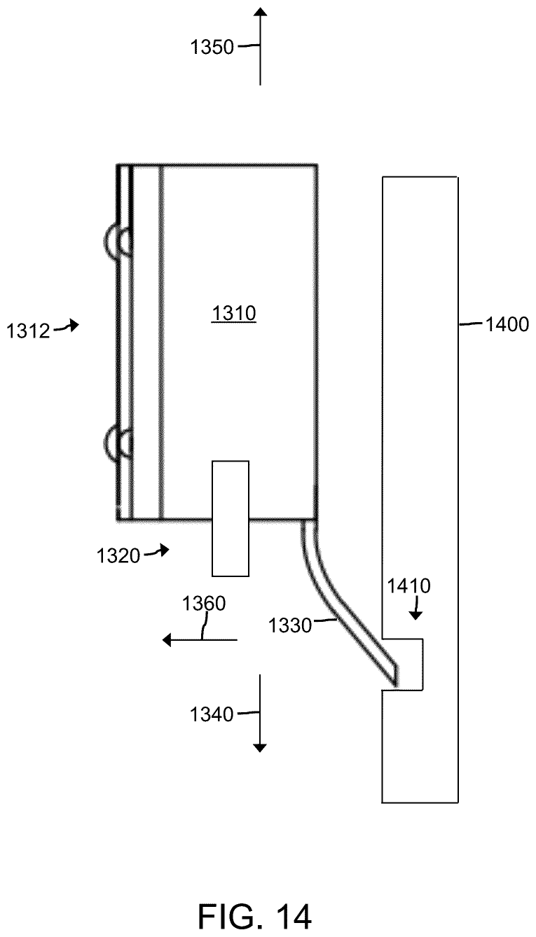

The tabs 1330 extend in a proximal direction 1340 away from the body 1310 and towards the fins 1400, for example as illustrated in FIG. 14, which is a side view of the clip 130. The tabs 1330 are configured to be mechanically engaged within and/or inserted into the holes 1410 in the fins 1400. The tabs 1330 can be flexible to allow the tabs 1330 to transition from one set of holes to an adjacent set of holes in the fins as the clip is moved in a distal direction 1350 relative to the fins and plunger. For example, the tabs 1330 are configured to flex in an upward direction 1360 away from the fins 1400 and holes 1410 (only one representative hole is illustrated in FIG. 14) when the tabs 1330 are inserted in one of the holes 1410 and the tabs 1330 are moved is the distal direction relative 1350 to the hole, which causes an edge of the hole to push the tab 1330 in the upward direction 1360. The upward direction 1360 is orthogonal to the proximal and distal directions 1340, 1350. The orientation of the tabs 1330 causes the tabs 1330 to resist flexing when a force is applied in the proximal direction 1340 so as to resist movement of the clip 1300 in the proximal direction 1340.

The clip 1300 can be formed of stainless steel (e.g., about 0.1 mm to about 0.3 mm in thickness such as about 0.2 mm in thickness), aluminum, plastic, and/or another material. The tabs 1330 can be formed of a thinner piece of material than the body 1310 to allow the tabs 1330 to be flexible. For example, the Young's modulus of the tabs 1330 can be smaller than the Young's modulus of the body 1310.

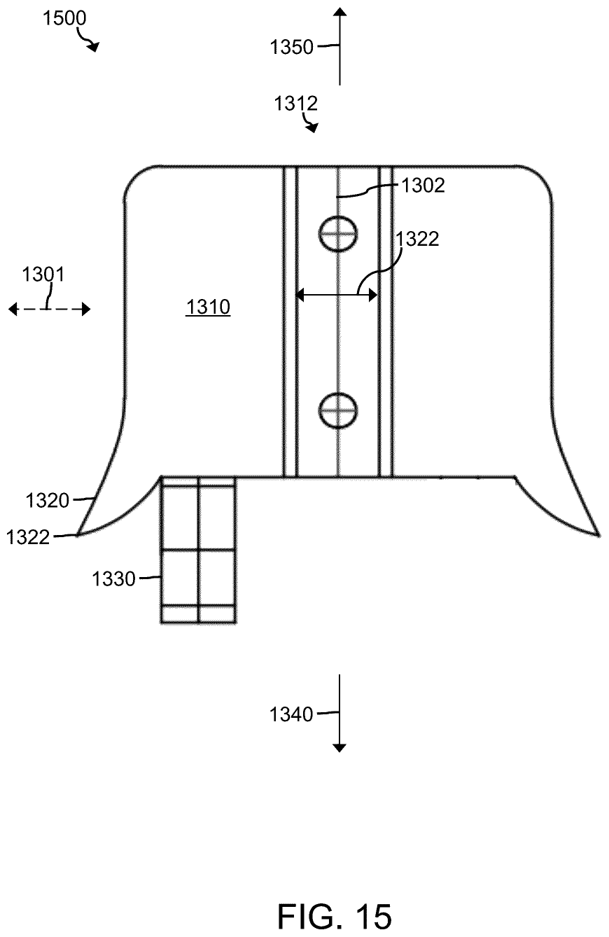

FIG. 15 is a bottom perspective view of a clip 1500 according to another embodiment. Clip 1500 is the same as clip 1300 except that clip 1500 has only one tab 1330 while clip 1300 has two tabs 1330. Clip 1500 is configured to be used with plunger 1200. Clip 1300 is configured to be used with plunger 800.

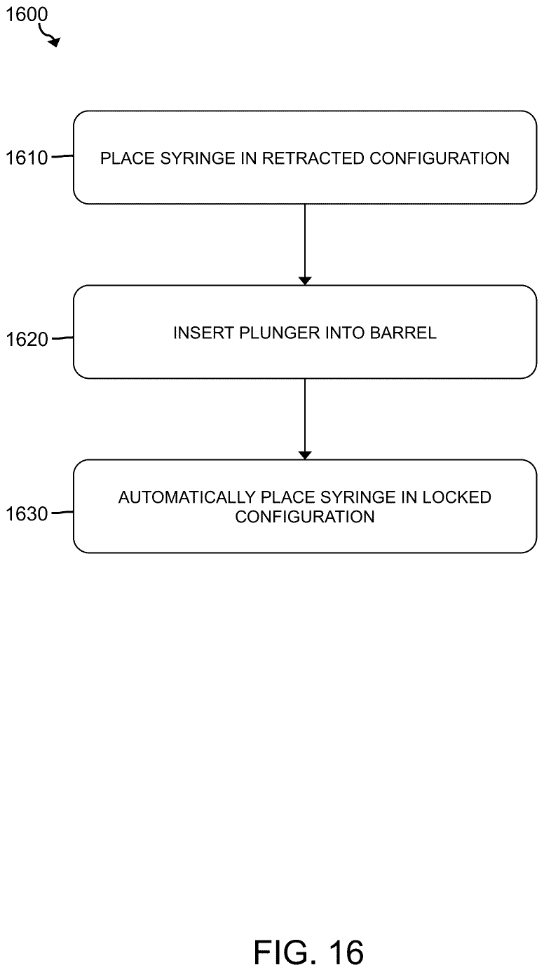

FIG. 16 is a flow chart of a method 1600 for using a single-use syringe according to an embodiment.

In step 1610, the syringe is placed in a retracted configuration. The retracted configuration can be a partially-retracted configuration (e.g., as illustrated in FIG. 2) or a fully-retracted configuration (e.g., as illustrated in FIG. 3). Placing the syringe is a retracted configuration can include applying a separation force between the plunger and the barrel of the syringe to transition the syringe from an initial configuration (e.g., as illustrated in FIG. 1) to the retracted configuration. The separation force can include a proximal force on the plunger with respect to the barrel to slide the plunger in a proximal direction with respect to the barrel. The clip is releasably mechanically coupled to the plunger as the plunger is retracted, for example due to the insertion and/or mechanical coupling of the tab(s) on the clip and the holes in the plunger fins.

The barbs on the clip, which are oriented in a proximal direction and outwardly towards the barrel wall, mechanically engage the barrel wall and resist a proximal movement of the clip within the barrel. The barbs are configured to slidably engage the barrel wall when the clip is moved distally within the barrel. The resistance of the proximal movement of the clip within the barrel causes the relative position of the clip and the plunger to change, such that the plunger moves in a proximal direction within the barrel while the clip remains in a fixed position within the barrel. As the relative position of the clip and the plunger changes, the tab(s) flex and mechanically engage different hole(s) in the plunger. When the plunger is fully retracted, the tab(s) mechanically engage the hole(s) at the distal end of the plunger. The plunger is partially retracted, the tab(s) mechanically engage one of the hole(s) between the distal and proximal ends of the plunger. As the plunger is retracted, fluid flows through the syringe tip into a fluid reservoir at the distal end of the barrel.

In step 1620, the plunger is partially or fully inserted into the barrel, such that the syringe is in a partially-inserted configuration or a fully-inserted configuration, for example as illustrated in FIGS. 3 and 4, respectively. The plunger can be inserted by applying an insertion force between the plunger and the barrel. The insertion force can cause the plunger to be inserted into the barrel. Since the clip is mechanically coupled to the plunger (e.g., via the tab(s) and hole(s) as discussed in step 1610), the insertion force causes a distal force to be applied to the plunger and the clip. The orientation of the barbs on the clip allows the clip to slidably engage the barrel wall, thereby allowing the clip to move in a distal direction in the barrel along with the plunger. As the plunger and a gasket attached to a distal end of the plunger are moved in the distal direction, fluid is expelled from the fluid reservoir. The fluid flows out of the syringe tip and through an optional needle attached thereto.

In the fully-inserted configuration, the gasket contacts and/or is compressed against the distal end of the barrel.

In step 1630, the syringe is automatically placed in a locked configuration when the plunger is partially or fully inserted into the barrel. The locked configuration is provided by the mechanical coupling of the clip and the plunger (e.g., by insertion and/or mechanical engagement of the tab(s) in the clip and the hole in the plunger) and the configuration of the barbs on the clip which resist proximal movement in the barrel. In the locked configuration, the plunger and the clip resist being retracted within the barrel, which would not allow the syringe to be reused.

When the plunger was partially retracted in step 1610, the tabs are mechanically engaged within and/or inserted one of the hole(s) between the distal and proximal ends of the plunger. The tabs remain engaged within this hole(s) in steps 1620 and 1630. When a separation force is applied between the plunger and the barrel, in an attempt to improperly reuse the syringe, the tabs translate the proximal force on the plunger to the clip. The barbs on the clip resist a proximal movement of the clip within the barrel. The resistance of the proximal movement of the clip within the barrel causes the relative position of the clip and the plunger to change, such that the plunger moves in a proximal direction within the barrel while the clip remains in a fixed position within the barrel. The plunger moves in the proximal direction until the tab(s) engage the hole(s) at the distal end of the plunger. When the tabs(s) engage the hole(s) at the distal end of the plunger, the flange at the distal end of the plunger contacts the distal end of the clip to prevent any further distal movement of the clip with respect to the plunger. Stated another way, the flange prevents any further proximal movement of the plunger with respect to the clip. The separation force is thus applied to both the plunger and the clip, and the separation force is resisted by the barbs, thus locking any further proximal movement of the plunger within the barrel.

When the plunger was fully retracted in step 1610, the tabs are mechanically engaged within and/or inserted the hole(s) at the distal end of the plunger. The tabs remain engaged within this hole(s) in steps 1620 and 1630. When the tabs(s) engage the hole(s) at the distal end of the plunger, the flange at the distal end of the plunger contacts the distal end of the clip to prevent any further distal movement of the clip with respect to the plunger. Stated another way, the flange prevents any further proximal movement of the plunger with respect to the clip. Any separation is thus applied to both the plunger and the clip, and the separation force is resisted by the barbs, thus locking any proximal movement of the plunger within the barrel.

In some embodiments, the resistance in the distal movement of the plunger within the barrel when the syringe is the locked state causes the plunger to break at a predetermined location. The predetermined location can be a predetermined point or region of mechanical weakness in the syringe. The breaking of the plunger can cause the syringe to be unusable.

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A single-use syringe comprising:
a hollow cylindrical barrel having a proximal end and a distal end, the distal end having a narrowed tip opening;
a plunger inserted through the proximal end of the hollow cylindrical barrel, the plunger comprising:
a plurality of longitudinal fins that extend radially from an axis, the longitudinal fins extending to a proximal end of the plunger, each longitudinal fin including a respective major planar surface, the longitudinal fins including at least first, second, and third fins, wherein:
the major planar surface of the first fin is aligned with the major planar surface of the second fin such that the major planar surfaces of the first and second fins lie in a common plane,
the major planar surface of the third fin is orthogonal to the common plane,
a first row of holes is defined in the major planar surface of the first fin,
a second row of holes is defined in the major planar surface of the second fin, and
the first and second rows of holes extend parallel to the axis;
a gasket attached to a distal end of the plunger; and
a clip comprising:
a body;
a slot defined in the body, the slot configured to slidably engage a radial edge of the third fin;
a pair of tabs configured to mechanically engage a pair of holes in the first and second rows of holes; and
a pair of barbs at a proximal end of the clip, the barbs oriented away from the body and in a proximal direction, the barbs configured to mechanically engage an internal surface of the hollow cylindrical barrel to resist a movement of the clip in a proximal direction with respect to the hollow cylindrical barrel and to slidably engage the internal surface of the hollow cylindrical barrel to allow a movement of the clip in a distal direction with respect to the hollow cylindrical barrel.

2. The syringe of claim 1, wherein:
the axis is a first axis, and
the holes in the first and second rows are evenly spaced and aligned with respect to a respective second axis that is orthogonal to the first axis.

3. The syringe of claim 2, wherein a position of the holes in the first and second rows corresponds to a predefined volume of fluid in the syringe.

4. The syringe of claim 1, wherein the gasket includes a gasket tip that is shaped to be inserted into the narrowed tip opening of the hollow cylindrical barrel.

5. The syringe of claim 1, wherein the syringe has a first configuration in which:
the plunger is fully inserted into the hollow cylindrical barrel,
the tabs are mechanically engaged with a pair of proximal end holes at a proximal end of the first and second rows of holes, and
the clip is located between the distal and proximal ends of the hollow cylindrical barrel.

6. The syringe of claim 5, wherein the syringe has a second configuration, the second configuration occurring after the first configuration, wherein in the second configuration:
the plunger is partially retracted from the hollow cylindrical barrel,
a position of the clip with respect to the hollow cylindrical barrel in the first and second configurations is the same,
a position of the clip with respect to the plunger is closer to the distal end of the plunger in the second configuration than in the first configuration,
the tabs are mechanically engaged with a respective pair of holes that correspond to the position of the clip with respect to the plunger, and
a fluid reservoir is defined between the gasket and the distal end of the hollow cylindrical barrel, the fluid reservoir holding a fluid.

7. The syringe of claim 6, wherein the syringe has a third configuration, the third configuration occurring after the second configuration, wherein in the third configuration:
the plunger is fully retracted from the hollow cylindrical barrel,
the position of the clip with respect to the hollow cylindrical barrel in the first, second, and third configurations is the same,
the position of the clip with respect to the plunger is closer to the distal end of the plunger in the third configuration than in the second configuration, the clip positioned adjacent to a flange at the distal end of the plunger,
the tabs are mechanically engaged with a pair of distal end holes at a distal end of the first and second rows of holes, and
a volume of the fluid reservoir is larger in the third configuration than in the second configuration.

8. The syringe of claim 7, wherein the syringe has a fourth configuration, the fourth configuration occurring after the third configuration, wherein in the fourth configuration:
the plunger is partially inserted into the hollow cylindrical barrel,
the tabs are mechanically engaged with the pair of distal end holes,
the position of the clip with respect to the hollow cylindrical barrel is closer to the distal end of the hollow cylindrical barrel in the fourth configuration than in the first, second, and third configurations, and
the volume of the fluid reservoir is smaller in the fourth configuration than in the third configuration such that some of the fluid flows out of the fluid reservoir through the narrowed tip opening of the hollow cylindrical barrel.

9. The syringe of claim 8, wherein the syringe has a fifth configuration, the fifth configuration occurring after the third or fourth configuration, wherein in the fifth configuration:
the plunger is fully inserted into the hollow cylindrical barrel,
the tabs are mechanically engaged with the pair of distal end holes,
the clip is positioned adjacent to the flange at the distal end of the plunger, and
the gasket is in physical contact with the distal end of the hollow cylindrical barrel, a position of the gasket causing the fluid to flow out of the fluid reservoir through the narrowed tip opening of the hollow cylindrical barrel.

10. The syringe of claim 9, wherein the fifth configuration is a locked configuration in which:
the flange prevents the clip from sliding in the distal direction with respect to the plunger,
the pair of barbs resists the movement of the clip in the proximal direction with respect to the hollow cylindrical barrel,
the position of the gasket prevents a movement of the plunger in the distal direction with respect to the hollow cylindrical barrel, and
a mechanical engagement of the tabs with the pair of distal end holes causes a resistance in the movement of the plunger in the proximal direction with respect to the hollow cylindrical barrel.

11. The syringe of claim 10, wherein the syringe has a sixth configuration, the sixth configuration occurring after the fifth configuration, wherein in the sixth configuration the plunger is broken at a predetermined mechanical failure point of the plunger, the break caused by a distal force on the plunger.

12. The syringe of claim 1, wherein an orientation notch is defined in the major planar surface of the third fin, the orientation notch configured to optically distinguish the third fin from the first and second fins.

13. The syringe of claim 1, wherein:
the common plane is a first plane, and
the longitudinal fins include a fourth fin, the major planar surface of the fourth fin aligned with the major planar surface of the third fin such that the major planar surfaces of the third and fourth fins lie in a second plane that is orthogonal to the first plane.

14. The syringe of claim 13, wherein:
an orientation notch is defined in the major planar surface of the third fin,
an orientation gap is defined in the major planar surface of the fourth fin, and
the orientation notch and the orientation gap are configured to optically distinguish the third and fourth fins, respectively, from the first and second fins and to optically distinguish the third fin from the fourth fin.

15. The syringe of claim 1, wherein the tabs extend in the proximal direction and towards the first and second fins.

16. A method of using a single-use syringe, comprising:
placing the syringe in a fully retracted configuration, the syringe comprising:
a hollow cylindrical barrel having a proximal end and a distal end, the distal end having a narrowed tip opening;
a plunger inserted through the proximal end of the hollow cylindrical barrel, the plunger comprising:
a plurality of longitudinal fins that extend radially from an axis, the longitudinal fins extending to a proximal end of the plunger, each longitudinal fin including a respective major planar surface, the longitudinal fins including at least first, second, and third fins, wherein:
the major planar surface of the first fin is aligned with the major planar surface of the second fin such that the major planar surfaces of the first and second fins lie in a common plane,
the major planar surface of the third fin is orthogonal to the common plane,
a first row of holes is defined in the major planar surface of the first fin,
a second row of holes is defined in the major planar surface of the second fin, and
the first and second rows of holes extend parallel to the axis;
a gasket attached to a distal end of the plunger; and
a clip comprising:
a body;
a slot defined in the body, the slot configured to slidably engage a radial edge of the third fin;
a pair of tabs configured to mechanically engage a pair of holes in the first and second rows of holes; and
a pair of barbs at a proximal end of the clip, the barbs oriented away from the body and in a proximal direction, the barbs configured to mechanically engage an internal surface of the hollow cylindrical barrel to resist a movement of the clip in a proximal direction with respect to the hollow cylindrical barrel and to slidably engage the internal surface of the hollow cylindrical barrel to allow a movement of the clip in a distal direction with respect to the hollow cylindrical barrel;
wherein in the fully-retracted configuration:
the plunger is fully retracted from the hollow cylindrical barrel,
the clip is located between the distal and proximal ends of the hollow cylindrical barrel,
the clip is positioned adjacent to a flange at the distal end of the plunger,
the tabs are mechanically engaged within a pair of distal end holes at a distal end of the first and second rows of holes, and
a fluid reservoir is defined between the gasket and the distal end of the hollow cylindrical barrel, the fluid reservoir holding a fluid; and
partially or fully inserting the plunger into the hollow cylindrical barrel; and
while partially or fully inserting the plunger:
flowing the fluid out of the fluid reservoir through the narrowed tip opening of the hollow cylindrical barrel;
maintaining a mechanical engagement of the tabs with the pair of distal end holes; and
sliding the barbs in the distal direction along the internal surface of the hollow cylindrical barrel,
wherein the mechanical engagement of the tabs within the pair of distal end holes and an orientation of the barbs cause the syringe to transition automatically to a locked configuration after an insertion of the plunger into the hollow cylindrical barrel.

17. The method of claim 16, further comprising, after the syringe is in the locked configuration:
applying a separation force between the plunger and the hollow cylindrical barrel;
mechanically engaging the barbs and the internal surface of the hollow cylindrical barrel, while applying the separation force, to resist a proximal movement of the clip with respect to the hollow cylindrical barrel; and
mechanically engaging the tabs within the pair of distal end holes, while applying the separation force, to resist a proximal movement of the plunger with respect to the hollow cylindrical barrel.

18. The method of claim 17, wherein applying the separation force causes the plunger to break at a predetermined mechanical weakness point.

19. The method of claim 16, further comprising:
retracting the plunger from a fully-inserted position to the fully-retracted configuration;
mechanically engaging the barbs and the internal surface of the hollow cylindrical barrel, while retracting the plunger, to resist a proximal movement of the clip with respect to the hollow cylindrical barrel;
flexing the tabs, while retracting the plunger, to mechanically disengage the tabs from a pair of proximal end holes at a proximal end of the first and second rows of holes;
sliding the radial edge of the third fin in the slot of the clip, while retracting the plunger, so that the tabs mechanically engage the pair of distal end holes; and
flowing the fluid into the fluid reservoir, while retracting the plunger, from the narrowed tip opening.

20. The method of claim 19, further comprising, while retracting the plunger:
mechanically engaging the tabs with a plurality of pairs of holes between the pair of proximal end holes and the pair of distal end holes; and
flexing the tabs to mechanically disengage the tabs from each pair of holes between the pair of proximal end holes and the pair of distal end holes.

21. A single-use syringe comprising:
a hollow cylindrical barrel having a proximal end and a distal end, the distal end having a narrowed tip opening;
a plunger inserted through the proximal end of the hollow cylindrical barrel, the plunger comprising:
a plurality of longitudinal fins that extend radially from an axis, the longitudinal fins extending to a proximal end of the plunger, each longitudinal fin including a respective major planar surface, the longitudinal fins including at least first, second, and third fins, wherein:
the major planar surface of the first fin is aligned with the major planar surface of the second fin such that the major planar surfaces of the first and second fins lie in a common plane,
the major planar surface of the third fin is orthogonal to the common plane,
a row of holes is defined in the major planar surface of the first fin, and
the row of holes extends parallel to the axis;
a gasket attached to a distal end of the plunger; and
a clip comprising:
a body;
a slot defined in the body, the slot configured to slidably engage a radial edge of the third fin;
a tab configured to mechanically engage a pair of holes in the first and second rows of holes; and
a pair of barbs at a proximal end of the clip, the barbs oriented away from the body and in a proximal direction, the barbs configured to mechanically engage an internal surface of the hollow cylindrical barrel to resist a movement of the clip in a proximal direction with respect to the hollow cylindrical barrel and to slidably engage the internal surface of the hollow cylindrical barrel to allow a movement of the clip in a distal direction with respect to the hollow cylindrical barrel.
